(19) United States
(12) Patent Application Publication
(10) Pub. No.: US 2011/0264114 A1
(43) Pub. Date: Oct. 27, 2011

(54) SURGICAL INSTRUMENT

(76) Inventors: Seung Wook CHOI, Seongnam-si (KR); Jong Seok WON, Yongin-si (KR); Jae Sun LEE, Seongnam-si (KR)

(21) Appl. No.: 13/176,446

(22) Filed: Jul. 5, 2011

Related U.S. Application Data
(62) Division of application No. 13/129,334, filed on May 13, 2011.

(30) Foreign Application Priority Data
Dec. 12, 2008 (KR) 10-2008-0126415
Dec. 30, 2008 (KR) 10-2008-0136840
Dec. 30, 2008 (KR) 10-2008-0136859
Jan. 21, 2009 (KR) 10-2009-0004872

(51) Int. Cl. A61B 19/00

(52) U.S. Cl. 606/130

(57) ABSTRACT

A surgical instrument is disclosed. The surgical instrument, which has an effector for engaging the surgical site joined to one end and a driving part for operating the effector joined to the other end, includes: a first shaft, which has one end joined with the driving part, and which extends along a first lengthwise direction; and a second shaft, which extends along a second lengthwise direction that forms a particular angle with the first shaft, and which has one end joined with the other end of the first shaft such that the second shaft is rotatable about an axis following the second lengthwise direction. Thus, it is possible to conduct surgery using several of such surgical instruments without having the instruments obstruct one another, and the surgical instrument can be made to have different usage modes according to what length it is set to.

### CROSS-REFERENCE TO RELATED APPLICATIONS

This application is a Divisional Application of co-pending U.S. patent application Ser. No. 13/129,334 filed May 13, 2011, which is the National Phase of PCT/KR2009/007290 filed on Dec. 8, 2009, which claims priority under 35 U.S.C. 119(a) to Patent Application No. 10-2008-0126415 filed in the Republic of Korea on Dec. 12, 2008, Patent Application No. 10-2008-0136859 filed in the Republic of Korea on Dec. 30, 2008, Patent Application No. 10-2008-0136840 filed in the Republic of Korea on Dec. 30, 2008, and Patent Application No. 10-2009-0004872 filed in the Republic of Korea on Jan. 21, 2009. All of those applications are hereby expressly incorporated by reference into the present application.

### BACKGROUND

The present invention relates to a medical apparatus, more particularly to a surgical instrument.

In the field of medicine, surgery refers to a procedure in which a medical apparatus is used to make a cut or an incision in or otherwise manipulate a patient's skin, mucosa, or other tissue, to treat a pathological condition.

Among existing types of surgery, a laparotomy is a surgical procedure in which the skin of the face or the abdomen is cut open and an internal organ, etc., is treated, reconstructed, or excised. When conducting a laparotomy, an incision is made in the skin and a particular amount of space is formed between the skin and the tissue, with the surgical operation performed within this space. As this may increase scars and prolong the healing period, laparoscopic surgery is recently gaining attention as an alternative.

In laparoscopic surgery, a small incision is made in the surgical site of the patient, through which a laparoscope is inserted, so that the surgery may be conducted while observing the surgical site within the abdominal cavity. Laparoscopic surgery is widely used in various fields of medicine, including internal medicine, surgery, urology, gynecology, and obstetrics. When conducting laparoscopic surgery, a surgical instrument may be used in which an effector is joined to the far end of a shaft that extends from the driving part. The effector portion of the surgical instrument may be inserted into the surgical site, and as the surgeon manipulates the driving part, the effector may function as a surgical tool, such as a set of scissors or forceps, etc., to perform various actions required for surgery.

The surgical instrument can be manipulated manually by the surgeon, or in the case of robot surgery, can be mounted onto the end portion of a robot arm, to be manipulated according to driving forces transferred from the robot arm.

However, in the case of a conventional instrument used for laparoscopic surgery, there is a limit to how small the diameter of the shaft can be made, and a trocar may have to be inserted beforehand into the surgical site through which to insert the instrument. In order to insert the trocar, the skin of the patient may have to be cut by a certain amount.

The information in the background art described above was obtained by the inventors for the purpose of developing the present invention or was obtained during the process of developing the present invention. As such, it is to be appreciated that this information did not necessarily belong to the public domain before the patent filing date of the present invention.

### SUMMARY

An aspect of the present invention is to provide a surgical instrument, and a method of setting the instrument, with which the diameter of the surgical instrument can be minimized, so that the incision made in the skin is of a size that does not require suturing.

Other technical problems addressed by the present invention will be readily understood from the descriptions that follow.

One aspect of the present invention provides a surgical instrument that includes: a driving part; a multiple number of rods joined to the driving part that extend along a lengthwise direction; and an effector detachably joined to a far end of the rods that operates in correspondence with a manipulation on the driving part. The multiple number of rods can form a set, which may be used as a shaft.

Another aspect of the present invention provides a surgical instrument that includes: a driving part; a multiple number of rods joined to the driving part that extend along a lengthwise direction and form a set serving as a shaft; and an effector that is joined to a far end of the rods and configured to operate in correspondence with a manipulation on the driving part. The effector can be detachably joined to the far end of the set of rods. In forming a set, the multiple number of rods can be fastened together at one or more points along the middle portion, or be twisted around one another. One or more of the rods can connect the driving part with the effector, serving to support and secure the effector in a particular position.

A multiple number of drivers, which may be joined respectively to one end of the rods, can be installed in the driving part, and the rods can be operated in correspondence with a manipulation on the respective drivers. The effector can include a multiple number of interlocking parts that are detachably joined with the other end of the rods, respectively, and the effector can be operated according to an operation of the rods while the rods are joined to the interlocking parts.

The interlocking parts are included in numbers that enable the effector to be operated with n (n is a natural number) degrees of freedom, the drivers are included in numbers that enable the driving part to be manipulated with n degrees of freedom, and the plurality of interlocking parts are joined by the plurality of rods with the plurality of drivers respectively in a corresponding manner.

In this case, an interlocking part and the other end of a rod can be shaped as a pair of linking devices that mate with each other. The multiple linking devices formed on the multiple interlocking parts and the other ends of the multiple rods can be formed with different shapes, so that each of the interlocking parts may be joined only with the other end of the rod which mates with the interlocking part.

A needle for invading can be mounted on the other end of the rod, while the rod can include a conductive element and an insulative element that coats and surrounds the conductive element, and the needle can be electrically connected with the conductive element. A cable can be used to supply electrical power to a tip part of the effector, which may then be used as an electrosurgical device.

The effector can be formed in a size capable of passing through a trocar inserted in the surgical site.

Another aspect of the present invention provides a method of setting a surgical instrument that includes: providing an effector, which includes a multiple number of interlocking parts, and which is configured to operate in accordance with a manipulation on the interlocking parts; providing a multiple number of rods, where each of the rods has one end joined to the driving part, the rods are configured to operate in correspondence with a manipulation on the driving part, and the rods each have a linking device formed on the other end in a shape that mates with a respective interlocking part; joining the other ends of the rods with the mating interlocking parts, respectively; and operating the effector by manipulating the driving part.

Additional aspects, features, and advantages, other than those described above, will be obvious from the claims and written description below.

Certain embodiments of the present invention make it possible to conduct surgery using several surgical instruments without having the instruments obstruct one another, and a surgical instrument can be made to have different usage modes according to what length it is set to.

Also, by forming the effector and a rod to be attachable and detachable in relation to each other, and by forming a multiple number of rods as one set that can be used as a substitute for the shaft, it is possible to minimize the diameter of the instrument. Furthermore, by first inserting the effector into the surgical site, then invading the rod and joining the effector and the rod within the surgical site, it is possible to utilize the surgical instrument after making an incision in the surgical site of a size that does not require suturing.

### BRIEF DESCRIPTION OF THE DRAWINGS

FIG. 1 is a diagram schematically illustrating a surgical instrument according to an embodiment of the present invention.

FIG. 2 is a lateral cross-sectional view of a set of rods according to an embodiment of the present invention.

FIG. 3 is a diagram schematically illustrating a surgical instrument according to another embodiment of the present invention.

FIG. 4 is a diagram schematically illustrating the driving part of a surgical instrument according to an embodiment of the present invention.

FIG. 5 is a diagram schematically illustrating the effector of a surgical instrument according to an embodiment of the present invention.

FIG. 6 is a diagram illustrating the operation of a surgical instrument according to an embodiment of the present invention.

FIG. 7 is a flowchart illustrating a method of setting a surgical instrument according to an embodiment of the present invention.

### DETAILED DESCRIPTION

As the present invention allows for various changes and numerous embodiments, particular embodiments will be illustrated in the drawings and described in detail in the written description. However, this is not intended to limit the present invention to particular modes of practice, and it is to be appreciated that all changes, equivalents, and substitutes that do not depart from the spirit and technical scope of the present invention are encompassed in the present invention.

While terms including ordinal numbers, such as “first” and “second,” etc., may be used to describe various components, such components are not limited to the above terms. The above terms are used only to distinguish one component from another. For example, a first component can be referred to as a second component without departing from the scope of claims of the present invention, and likewise, a second component can be referred to as a first component. If a component is said to be “connected to” or “accessing” another component, it is to be appreciated that the two components can be directly connected to or directly accessing each other but can also include one or more other components in-between.

The terms used in the present specification are merely used to describe particular embodiments, and are not intended to limit the present invention. An expression used in the singular encompasses the expression of the plural, unless it has a clearly different meaning in the context. In the present specification, it is to be understood that the terms “including” or “having,” etc., are intended to indicate the existence of the features, numbers, steps, actions, components, parts, or combinations thereof disclosed in the specification, and are not intended to preclude the possibility that one or more other features, numbers, steps, actions, components, parts, or combinations thereof may exist or may be added.

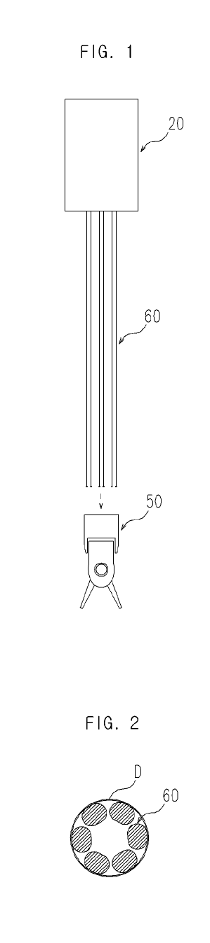

FIG. 1 is a diagram schematically illustrating a surgical instrument according to an embodiment of the present invention, and FIG. 2 is a lateral cross-sectional view of a set of rods according to an embodiment of the present invention. Illustrated in FIG. 1 and FIG. 2 are a driving part 20, rods 60, and an effector 50.

A feature of this embodiment is embodiment is that the effector 50 and the rods 60 of the surgical instrument are configured to be attachable and detachable in relation to each other, so that the rods 60 can be joined to the effector 50 after first inserting the separately detached effector 50 into the abdominal cavity and then invading the rods 60. Thus, the surgical instrument can be manipulated for surgery after making an incision that is small enough not to leave a scar.

The instrument according to this embodiment may be composed mainly of a driving part 20, a multiple number of rods 60 joined to the driving part 20, and an effector 50 detachably joined to the far end of the rods 60. The driving part 20 is a part that can be manually operated by a surgeon in the case of manual operation, and can be manipulated by driving forces transferred from the robot arm in the case of robotic surgery.

The rods 60 are components that may move along a particular lengthwise direction according to a manipulation on the driving part 20. For example, in cases where multiple driving wheels are mounted on the driving part 20 and the rods 60 are pulley-joined to the driving wheels, respectively, each rod 60 may serve to transfer a tensional force according to the rotation of the driving wheel. Alternatively, drivers that each perform a reciprocating movement along the lengthwise direction of the rods 60 can be mounted instead of the driving wheels, and the rods 60 can be joined to the drivers, in which case the rods 60 can be made to transfer forces along the lengthwise direction when the drivers are manipulated.

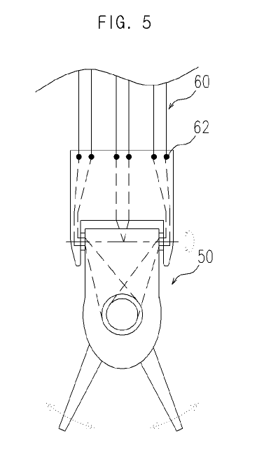

The effector 150 is the component that is actually inserted into the surgical site to perform a gripping or cutting movement, etc. The effector 50 according to this embodiment may be joined to the far end of the rods 60, configured such that the effector 50 can be separated from the rods 60 and the separated effector 50 can be reattached to the rods 60 as necessary. The linking structure between the rods 60 and the effector 50 will be described in further detail in the paragraphs describing FIG. 5.

When the effector 50 is thus joined to the far end of the rods 60, the rods 60 may transfer forces according to the manipulation of the driving part 20, causing the parts of the effector 50 to operate. As a result, the effector 50 may perform a gripping or cutting motion.

When using a surgical instrument according to this embodiment, the detached effector 50 may first be inserted into the surgical site, and then the rods 60 may invade the surgical site, after which the effector 50 may be joined to the far end of the rods 60 inside the surgical site and manipulated. Therefore, the separately detached effector 50 may be formed in a size that can be inserted into the surgical site, i.e. a size that allows the effector 50 to pass through a trocar inserted at the surgical site.

In this way, the effector 50 may be inserted through a trocar, which itself is inserted beforehand, and the instrument may invade the surgical site directly, where the effector 50 may afterwards be joined to the rods 60 inside the surgical site.

Furthermore, an instrument according to this embodiment can employ a set of rods 60 instead of a separate shaft member, as illustrated in FIG. 2, so that the diameter of the shaft (i.e. the set of rods 60) can be minimized. For example, if the diameter of the set of rods 60 is set to 2 mm or smaller, similar to the diameter of a syringe needle, then there is no need to suture the skin and there is no scar left behind in the skin after the rods 60 invade the skin of the surgical site, so that the laparoscopic surgery may be performed with a greater level of safety.

Thus, the multiple number of rods 60 according to this embodiment can form a set which itself may serve as the shaft, while one or more bands or rings (see “D” in FIG. 2) for binding the rods can be placed intermittently on the multiple number of rods.

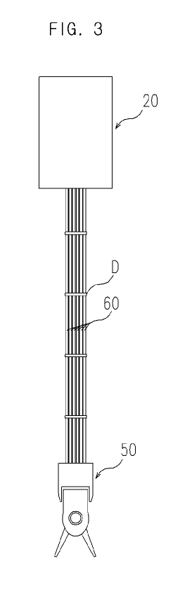

FIG. 3 is a diagram schematically illustrating a surgical instrument according to another embodiment of the present invention. Illustrated in FIG. 3 are a driving part 20, rods 60, and an effector 50.

This embodiment provides an example of a surgical instrument that uses a set of multiple rods as a substitute for the shaft. The main composition of a driving part 20, a multiple number of rods 60 joined to the driving part 20, and an effector 50 detachably joined to the far end of the rods 60 is substantially the same as that of the previously described embodiment. As the functions, structures, and operating methods of the driving part 20 and the effector 50 are substantially the same as those of the previously described embodiment, details on this matter will be omitted here.

The multiple number of rods 60 according to this embodiment may form a set, to function as the “shaft” extending in a lengthwise direction. In other words, instead of using a separate shaft, a set of rods 60 can be used, with the several rods 60 gathered together, fastened together with bands or rings, etc., (see “D” in FIG. 3) in intervals along the middle to prevent the bundle of rods from being separated, or even twisted around one another to form a set. In this way, the diameter of the instrument can be minimized, and the surgical instrument can be used after making an incision in the surgical site that is small enough not to require suturing.

Of course, it is not imperative that the effector 50 and the rods 60 be connected to each other in implementing this embodiment, and a detachably attachable structure can be employed between the far end of the set of rods 60 and the effector 50, similar to the previously described embodiment.

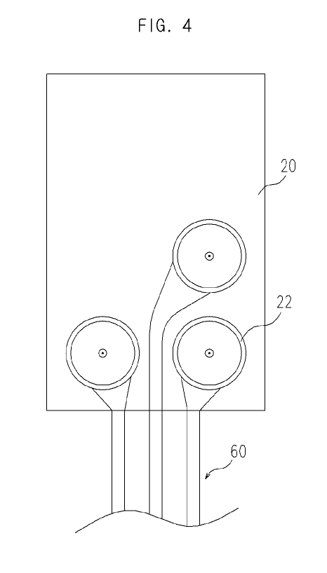

FIG. 4 is a diagram schematically illustrating the driving part of a surgical instrument according to an embodiment of the present invention. Illustrated in FIG. 4 are a driving part 20, drivers 22, and rods 60.

The embodiment shown in FIG. 4 illustrates an example in which there are a multiple number of wheel-shaped drivers 22 arranged in the driving part 20, where a pair of rods 60 are joined to each of the drivers 22. As described above, various joining methods can be applied, such as pulley-joining the rods 60 to the drivers 22 and joining one end of each rod 60 to a portion of a driver 22.

When the wheel-shaped driver 22 is rotated, the rods 60 joined to the driver 22 may move along the lengthwise direction, thereby transferring the driving force to a part of the effector 50 joined to the other end of the rods 60.

However, FIG. 4 is an illustration of just one example. It is not imperative that the drivers 22 be limited to wheel-like shapes, neither is it imperative that a pair of rods 60 be joined to each driver 22. The composition of the drivers 22 and rods 60 can be implemented using various structures, for example with one rod 60 joined to one driver 22 that undergoes a reciprocating movement along the lengthwise direction of the rod 60.

FIG. 5 is a diagram schematically illustrating the effector of a surgical instrument according to an embodiment of the present invention. Illustrated in FIG. 5 are rods 60, an effector 50, and interlocking parts 62.

The effector 50 illustrated in FIG. 5 comprises three movable parts, namely, a pair of claws, each of which may rotate about a particular rotational axis, and a tilting axis, about which the whole of the forceps may perform a tilting movement. Thus, the effector 50 according to this embodiment may move with 3 degrees of freedom.

To enable each part of the effector 50 to move or rotate, each movable part may include two interlocking parts 62. Referring to two interlocking parts 62 equipped to rotate a claw, for example, pulling on one of the interlocking parts 62 may move the claw to open, while pulling on the other may move the claw to close. For the tilting axis, pulling on one of the two interlocking parts 62 corresponding to the tilting axis may tilt the set of claws in a plus (+) direction, while pulling the other may tilt the claws in a minus (−) direction.

If two interlocking parts 62 are thus provided for each movable part, then an effector 50 according to this embodiment that has 3 degrees of freedom may include a total of six interlocking parts 62. To these interlocking parts 62, the other ends of the rods 60 described above may be joined, respectively, so that the tensional forces transferred through the respective rods 60 may enable the parts of the effector 50 to operate according to the manipulations on the driving part 20.

However, FIG. 5 is an illustration of just one example. It is not imperative that the effector 50 operate with 3 degrees of freedom, neither is it imperative that a pair of interlocking parts 62 be included for each movable part of the effector 50. The composition of the effector 50 and interlocking parts 62 can be implemented in various ways, for example with one interlocking parts 62 included for one movable part and with the movable part configured to operate according to the pulling or pushing of the corresponding interlocking part 62.

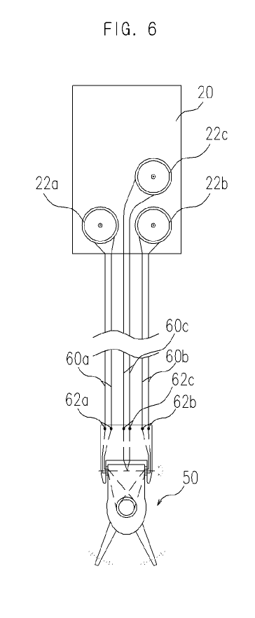

FIG. 6 is a diagram illustrating the operation of a surgical instrument according to an embodiment of the present invention. Illustrated in FIG. 6 are a driving part 20, drivers 22a, 22b, 22c, rods 60a, 60b, 60c, an effector 50, and interlocking parts 62a, 62b, 62c.

FIG. 6 illustrates an example of an instrument formed by joining the driving part 20 with the effector 50 as described above. A description will now be provided as follows on the operation of an instrument according to this embodiment, with reference to FIG. 6.

As already described above, if the effector 50 according to this embodiment is a structure that operates with n degrees of freedom (n is a natural number), then a multiple number of interlocking parts 62a, 62b, 62c may be included in correspondence with the movable parts of the effector 50. Looking at the assembly shown in FIG. 5, for example, the effector 50 may operate with 3 degrees of freedom, and there may be two interlocking parts 62a, 62b, 62c corresponding with each movable part, resulting in a total of six interlocking parts 62a, 62b, 62c.

On the other hand, the driving part 20 according to this embodiment may also be included in multiple numbers in correspondence with the degree of freedom of the effector 50. In cases where the effector 50 operates with 3 degrees of freedom, as is the case shown in FIG. 5, the driving part 20 may correspondingly include three drivers 22a, 22b, 22c, so that the driving part 20 may be manipulated with 3 degrees of freedom.

The multiple number of drivers 22a, 22b, 22c and interlocking parts 62a, 62b, 62c may be joined, respectively, to correspond with each other, by way of the multiple number of rods 60a, 60b, 60c, and the driving forces generated (or transferred) according to the manipulation of the drivers 22 may be transferred through the rods 60 to the effector 50, which may then perform various maneuvers required for surgery.

As described above, an instrument according to this embodiment may use a set of rods 60 instead of a separate shaft member, to minimize the diameter of the instrument.

While the multiple number of rods 60a, 60b, 60c are joined with the multiple number of interlocking parts 62a, 62b, 62c to correspond with each other, when one of the rods 60a is operated to move an interlocking part 62a, the other rods 60b, 60c and the interlocking parts 62b, 62c joined to these rods 60b, 60c may support the effector 50 such that the whole of the effector 50 does not move and remains secured to a particular position. Thus, when a rod 60a is operated, only the interlocking part 62a joined to the rod 60a may move.

It is also possible to install a separate securing rod (not shown) in the center or use one or some of the rods 60a, 60b, 60c as a securing rod. Then, the securing rod may support the effector 50 such that the whole of the effector 50 remains secured to a particular position without moving, while the remaining rods may operate to move the respective interlocking parts joined to the rods.

For the purpose of joining the other ends of the rods 60 to the multiple interlocking parts 62 included in the effector 50, a pair of linking devices that mate with each other can be formed on an interlocking part 62 and the other end of a rod 60. Various types of linking device can be applied, examples of which may include forming the interlocking part 62 as an indentation that includes a detent curb and forming the other end of the rod 60 as a hook that is inserted into the indentation and caught on the detent curb; forming the other end of the rod 60 and the interlocking part 62 as a joint, such as a tongue and groove joint, etc.; and attaching a pair of magnets to the other end of the rod 60 and the interlocking part 62.

According to this embodiment, each pair of an interlocking part 62 and a rod 60 may be joined corresponding with each other, and to this end, each pair of linking devices formed on the other end of a rod 60 and an interlocking part 62 can be formed with a different shape and/or structure for each rod 60 (each interlocking part 62). Considering an example where a first rod 60a is to be joined with a first interlocking part 62a and a second rod 60b is to be joined with a second interlocking part 62b, the linking device for the first rod and interlocking part can have a different shape and/or structure from the linking device for the second rod and interlocking part (for example, by forming the first linking device with a square cross section and forming the second linking device with a triangular cross section). Thus, each of the rods 60 and each of the interlocking parts 62 may mate with each other, i.e. a particular rod 60 may be joined with only its counterpart interlocking part 62.

However, it is not imperative that this joining of the interlocking parts 62a, 62b, 62c and rods 60a, 60b, 60c to correspond with each other be implemented by providing different shapes or structures for the linking devices. It is also possible to join the multiple interlocking parts 62a, 62b, 62c and rods 60a, 60b, 60c to each other randomly, and afterwards match the movable parts of the effector 50 with the respective drivers required for manipulation, by identifying which rod 60 is joined to which interlocking part 62.

Various methods can be applied for identifying which rod 60 is joined to which interlocking part 62, where some examples include a method of manually configuring the settings after the effector 50 is joined, and a method of forming electrical contacts on each of the interlocking parts 62 and having the driving part 20 check the ID's of the respective interlocking parts 62 from electrical signals transferred through the rods 60 joined to the interlocking parts 62.

In cases where an instrument according to this embodiment is mounted on a surgical robot for usage, the above matching between the effector 50 and the drivers 22 can be implemented using software within the system for driving the surgical robot. In such cases, the matching settings can be modified according to the requirements of the user, to reconfigure which part of the effector 50 will be operated by which driver 22.

To invade the skin of the patient, a rod 60 according to this embodiment can be formed such that its tip has a needle-like structure. In other words, a needle (not shown) for invasion can be mounted on the other end of the rod 60. If such is the case, a rod 60 according to this embodiment can be structured to have an insulative element coating a conductive element, with the tip of the conductive element electrically connected to a needle, in order that the rod may be used for transferring electrical signals, as described above, or in order that the rod may be used for electrosurgery, as described below.

A surgical instrument according to this embodiment can also be used for electrosurgery. That is, one or more cables (not shown) can be included in addition to the multiple number of rods 60 described above, where the cables may electrically connect the driving part 20 with the effector 50, so that the tip of the effector 50 may be used as an electrosurgical device.

An electrosurgical device can be utilized for stopping blood loss in a vein, cutting tissue, removing small polyps, etc., using a probe equipped with a metal cap or a metal wire that is heated to high temperatures by electricity, as well as for cutting or coagulating tissue using various types of RF waves.

For example, by adding a cable that includes a conductive element coated with an insulative element and electrically connecting the conductive element of the cable to the tip portion of the effector 50, the tip portion of the effector 50 can be supplied with electrical power from the cable to function as an electrosurgical device.

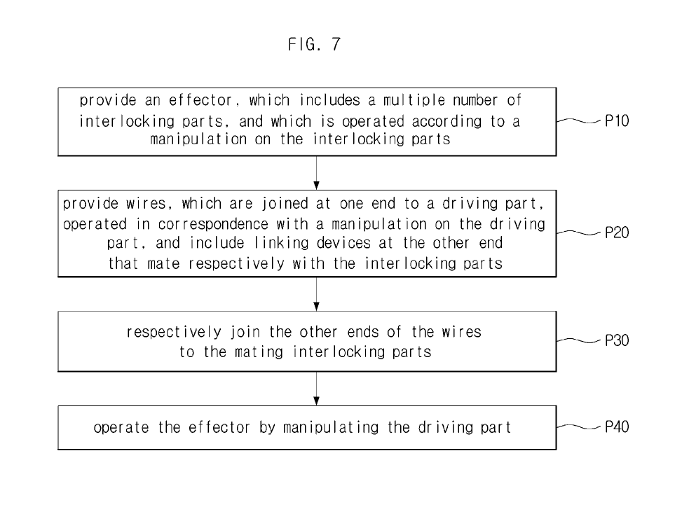

FIG. 7 is a flowchart illustrating a method of setting a surgical instrument according to an embodiment of the present invention. This embodiment relates to a method of setting the instrument described above for use on the surgical site.

First, a separate detached effector module 50 may be provided (P10). An effector 50 according to this embodiment can include a multiple number of interlocking parts 62, as described above, where each part of the effector 50 may be operated according to the manipulation on the interlocking parts 62.

Next, the body of the instrument may be provided, which includes a multiple number of rods 60 joined to the driving part 20 (P20). As already described above, the multiple number of rods 60 may be joined in one end to a multiple number of drivers 22, and each of the rods 60 may be operated in accordance with a manipulation on the respective driver 22.

Next, the other ends of the multiple rods 60 may be joined respectively to the multiple interlocking parts 62 (P30). As described above, the multiple rods 60 and multiple interlocking parts 62 may be joined respectively in correspondence with each other, and for this purpose, each pair of linking devices for a rod 60 and an interlocking part 62 can be formed with a different shape and/or structure for each rod 60, or the rods 60 and the interlocking parts 62 can be joined randomly and matched for correspondence afterwards.

After thus providing the effector module 50 and the instrument body separately and joining them together, the drivers 22 of the driving part 20 may be manipulated to operate the respective parts of the effector 50 (P40). In this way, the setting procedures may be completed for performing a maneuver required for surgery by manipulating the surgical instrument.

When the surgical instrument is set and used according to this embodiment, the separate, detached effector module 50 may be inserted into the surgical site, and the rods 60 may invade the surgical site, after which the effector 50 and the rods 60 may be joined inside the surgical site. Since the surgery can be conducted by inserting the instrument after making an incision in the surgical site of a size that does not require suturing, there may be no scar left in the surgical site, and the laparoscopic surgery may be performed with a greater level of safety.

While the present invention has been described with reference to particular embodiments, it will be appreciated by those skilled in the art that various changes and modifications can be made without departing from the spirit and scope of the present invention, as defined by the claims appended below.

What is claimed is:

1. A surgical instrument comprising:
a driving part operated by a user manipulation;
a plurality of drivers included in the driving part, the drivers configured to enable the driving part to be manipulated with n degrees of freedom (n is a natural number);
a plurality of rods extending along a lengthwise direction to form a set to serve as a shaft, one ends of the plurality of rods joined with the plurality of drivers respectively to move along the lengthwise direction respectively in correspondence with a manipulation on the plurality of drivers, the other ends of the plurality of rods invading a surgical site; and
an effector formed in a size capable of inserting into the surgical site, the effector including a plurality of interlocking parts each detachably joined with the other ends of the plurality of rods respectively in the surgical site, the effector operated according to the motion along the lengthwise direction of the rods while the rods are joined to the interlocking parts,
wherein the plurality of interlocking parts are joined by the plurality of rods with the plurality of drivers respectively in a corresponding manner;
a plurality of pairs of linking devices mating with each other are formed on the plurality of interlocking parts and the other ends of the plurality of rods such that any one of the plurality of interlocking parts is joined only with the other end of a mating rod; and
the effector is joined to the other ends of the plurality of rods for usage inside the surgical site.

2. The surgical instrument according to claim 1, wherein the plurality of rods are fastened together at one or more points along a middle portion thereof to form a set.

3. The surgical instrument according to claim 1, wherein the plurality of rods are twisted around one another to form a set.

4. The surgical instrument according to claim 1, wherein any one or more of the plurality of rods connect the driving part with the effector to support and secure the effector in a particular position.

5. The surgical instrument according to claim 1, wherein the plurality of linking devices formed on the plurality of interlocking parts and the other ends of the plurality of rods are formed with different shapes.

6. The surgical instrument according to claim 1, wherein a needle for invading is mounted on the other end of the rod.

7. The surgical instrument according to claim 6, wherein the rod comprises a conductive element transferring electrical signals and an insulative element coating and surrounding the conductive element, and the needle is electrically connected with the conductive element.

8. The surgical instrument according to claim 1, further comprising a cable electrically connecting the driving part and the effector.

9. The surgical instrument according to claim 8, wherein the cable comprises a conductive element and an insulative element coating and surrounding the conductive element, and the conductive element is electrically connected with a tip part of the effector.

10. The surgical instrument according to claim 8, wherein a tip part of the effector is supplied with electrical power from the cable to serve as an electrosurgical device.

11. The surgical instrument according to claim 1, wherein the effector is formed in a size capable of passing through a trocar inserted in a surgical site.

12. A method of setting a surgical instrument, the method comprising:
providing an effector, the effector including a plurality of interlocking parts and configured to operate in accordance with a manipulation on the interlocking parts;
providing a plurality of rods each having one end thereof joined to the driving part, the plurality of rods configured to operate in correspondence with a manipulation on the driving part, the plurality of rods each having a linking device formed on the other end thereof in a shape mating with the plurality of interlocking parts respectively;
joining the other ends of the plurality of rods with the plurality of mating interlocking parts respectively; and
operating the effector by manipulating the driving part.
